(19) United States
(12) Patent Application Publication
(10) Pub. No.: US 2026/0128130 A1
(43) Pub. Date: May 7, 2026

(54) CLASSIFIER AND TRAINING SYSTEM FOR EARLY IDENTIFICATION OF PLACENTA ACCRETA SPECTRUM RISK IN HIGH-RISK PREGNANT WOMEN

(71) Applicant: CHANGZHOU MATERNAL AND CHILD HEALTH CARE HOSPITAL, Jiangsu (CN)

(72) Inventors: Zhonghua SHI, Jiangsu (CN); Runrun HAO, Jiangsu (CN); Bin ZHANG, Jiangsu (CN); Xueqi BAI, Jiangsu (CN); Shanshan WANG, Jiangsu (CN); Shiman HU, Jiangsu (CN); Sutong KAN, Jiangsu (CN); Haoyan SHI, Jiangsu (CN)

(73) Assignee: CHANGZHOU MATERNAL AND CHILD HEALTH CARE HOSPITAL, Jiangsu (CN)

(21) Appl. No.: 19/344,570

(22) Filed: Sep. 30, 2025

(30) Foreign Application Priority Data
Sep. 30, 2024 (CN) 202411383048.5

(51) Int. Cl. G16B 40/00; C12Q 1/6883; G16B 30/10; G16H 50/30

(52) U.S. Cl. CPC G16B 40/00; C12Q 1/6883; G16B 30/10; G16H 50/30; C12Q 2600/118; C12Q 2600/158

(57) ABSTRACT

A classifier and a training system for early identification of a placenta accreta spectrum risk in high-risk pregnant women. This system implements normalization and discretization based on analysis with genome-wide maternal plasma cell-free DNA promoter coverages, and identifies an optimal target combination with 23 genes including ABHD1, ALG1L2, EYS, and the like, demonstrating potential for preparation of a diagnostic kit. Based on a machine learning algorithm, the constructed classifier exhibits high sensitivity and specificity in predicting occurrence of PAS, and areas under the receiver operating characteristic curve (AUC) all exceed 0.85, effectively implementing early risk assessment for high-risk pregnant women. This classifier clinically provides a non-invasive predictive tool, holding significant clinical application value and medical significance.

### CROSS-REFERENCE TO RELATED APPLICATION

This application claims the priority benefit of China application serial no. 202411383048.5, filed on Sep. 30, 2024. The entirety of the above-mentioned patent application is hereby incorporated by reference herein and made a part of this specification.

### BACKGROUND

### Technical Field

The present invention relates to the field of model prediction, and in particular, to a classifier and a training system for early identification of a placenta accreta spectrum risk in high-risk pregnant women.

### Background Art

Placenta accreta spectrum (PAS) is a leading cause of critical and life-threatening obstetric conditions. However, its prenatal diagnosis has limitations such as late detection and frequent missed diagnoses, posing significant challenges especially for primary hospitals. Studies indicate that two-thirds of PAS cases remain undiagnosed clinically. Failure to identify PAS prenatally is a risk factor for intrapartum and postpartum massive hemorrhage, blood transfusion, emergency interventions, and hysterectomy. Therefore, accurate early prediction of PAS helps high-risk pregnant women make informed reproductive decisions, facilitates high-risk referrals, allows for multidisciplinary consultations, and reduces risks for pregnant or lying-in women and perinatal infants.

Cell-free DNA (cfDNA) in plasma originates from the release of apoptotic cells. The cfDNA carries nucleosome footprints that can reflect gene expression information of its tissue of origin. During pregnancy, approximately 10% of cfDNA in circulating blood originates from the placenta. Therefore, cfDNA in plasma in early pregnancy carries gene expression information of the placenta and decidua. During early-to-mid pregnancy, genome-wide cfDNA promoter nucleosome coverage profiles can reflect expression patterns of tissue of origin, demonstrating extremely high predictive value for placenta-derived diseases, particularly PAS. Non-invasive prenatal testing (NIPT) is clinically common for prenatal screening, and relies on low-coverage whole-genome sequencing across different sequencing platforms for hospitals worldwide, such as Illumina, Life, and BGI. In recent years, NIPT-based extraction of cfDNA promoter nucleosome coverage profiles has shown significant value not only in screening for fetal chromosomal abnormalities, but also in early prediction of pregnancy complications, such as fetal growth restriction, macrosomia, and preeclampsia. However, there is no effective early prediction model for placenta accreta spectrum.

### SUMMARY

To address practical needs and drawbacks in the related art, the present invention provides a classifier and a training system for early identification of a PAS risk in high-risk pregnant women, to resolve current lack of methods for accurately predicting occurrence of PAS during early-to-mid pregnancy.

### Technical Solutions

To achieve the foregoing objective, the present invention provides the following technical solutions:

According to a first aspect, the present invention provides a classifier for early identification of a PAS risk in high-risk pregnant women based on plasma cfDNA promoter coverages, where a target gene combination includes ABHD1, ALG1L2, EYS, FAM157C, KDSR, KRT5, LANCL2, LINC00390, LINC00964, LOC105371998, LOC107987394, LOC644090, LYZL2, MIR184, MIR4802, MYT1L, NGDN, NSD2, PACRG.AS3, SAP30L.AS1, SLC16A12.AS1, TADA3, and TMEM147.AS1.

In the present invention, genome-wide cfDNA promoter coverage profiles are discovered in plasma of pregnant women with occurrence of PAS during early-to-mid pregnancy based on NIPT data, an optimal gene combination and an optimal cutoff value of each gene are obtained based on machine learning strategies (see a second aspect) to train an optimal classifier, and an area under the curve (AUC) of the receiver operating characteristic curve (ROC) is predicted in an independent validation dataset, which reaches 0.85 or more, demonstrating good potential as a screening means for PAS in high-risk pregnant women.

A method for assessing a placenta accreta spectrum risk in high-risk pregnant women includes the following steps:

Data collection and preprocessing: collect low-coverage whole-genome sequencing data of high-risk pregnant women undergoing NIPT, and perform necessary preprocessing, to ensure data quality.

Promoter region identification and coverage extraction: align NIPT data to human reference genome hg19 by using software bwa-mem, SAMtools, and BEDtools and a sequence alignment algorithm, and determine promoter regions pTSS from −1000 bp to +1000 bp around transcription start sites (TSS) of 23 genes: ABHD1, ALG1L2, EYS, FAM157C, KDSR, KRT5, LANCL2, LINC00390, LINC00964, LOC105371998, LOC107987394, LOC644090, LYZL2, MIR184, MIR4802, MYT1L, NGDN, NSD2, PACRG.AS3, SAP30L.AS1, SLC16A12.AS1, TADA3, and TMEM147.AS1, to obtain original read coverages of the 23 genes at the pTSS regions.

Feature factor normalization: normalize the original read coverages of the 23 genes at the pTSS regions by a TPM-like method, to obtain TPM-like normalized pTSS coverages (NPC-TPM) of the 23 genes.

$$NPC - {TPM}_{i} = \frac{{q}_{i} / {l}_{i}}{{\sum }_{j} ( {q}_{j} / {l}_{j} )} * 1 {0}^{6} = \frac{{q}_{i}}{{\sum }_{j} {q}_{j}} * 1 {0}^{6}$$

where NPC-TPMᵢ represents a TPM-like normalized pTSS coverage of a gene i, qᵢ represents an original read coverage at the pTSS region, lᵢ represents a transcript length (all being 2000), and Σⱼ(qⱼ/lⱼ) represents a sum of pTSS read coverages normalized based on the transcript length.

Feature factor discretization: compare the NPC-TPM values of the 23 genes respectively to optimal cutoff values (obtained in the second aspect) of all the genes. A case in which a feature factor NPC-TPM is greater than a corresponding optimal cutoff value is set to 1. A case in which a feature factor NPC-TPM is not greater than a corresponding optimal cutoff value is set to 0.

Risk assessment: input the discretized NPC-TPM values of the 23 genes of pregnant women to the classifier, to calculate a PAS risk.

This method is applicable to singleton pregnancy with at least one of the following high-risk factors of PAS: (1) history of uterine surgery, such as cesarean section, myomectomy, and uterine septum resection; (2) history of intrauterine procedures, such as hysteroscopic surgery and curettage; and (3) pregnancy achieved via in vitro fertilization-embryo transfer (IVF-ET).

This process provides a non-invasive assessment means through the extraction of the pTSS coverage based on the NIPT data. This indicates that a particular combination and an expression pattern of the 23 genes demonstrate significant potential for preparation of a diagnostic kit. During development of the diagnostic kit, the expression of these genes is detected by using an accurate molecular diagnosis technique. A detection method may be designed with high sensitivity and high specificity, to implement accurate early prediction for PAS high-risk pregnant women, with the potential to become a routine clinical detection item, serving a broader patient population.

According to a second aspect, the present invention provides a classifier training system for a PAS risk in high-risk pregnant women based on plasma cfDNA promoter sequencing during early-to-mid pregnancy.

A classifier training system for a PAS risk in high-risk pregnant women based on plasma cfDNA promoter sequencing during early-to-mid pregnancy includes the following modules:

A dataset division module is configured to extract pre-collected medical data of PAS high-risk pregnant women at NIPT on different sequencing platforms, match pregnant women with occurrence of PAS and pregnant women without occurrence of PAS based on maternal age, gestational age at NIPT, fetal sex, and distribution of high-risk factors, randomly divide samples from a primary platform into a training dataset and an internal validation dataset, and use samples from the remaining platform as an independent external validation dataset.

An impact factor extraction module is configured to perform annotation on pre-collected NIPT data of the PAS high-risk pregnant women with cfDNA promoter nucleosome coverage profiles and perform feature extraction.

The NIPT is low-depth high-throughput sequencing on cell-free DNA in maternal peripheral blood. The NIPT data is aligned to human reference genome hg19 by using an accurate sequence alignment algorithm and using software bwa-mem, SAMtools, and BEDtools, PCR duplicates are removed, a region from −1000 bp to +1000 bp around a transcription start site (TSS) is determined as a promoter region pTSS, and an original read coverage at the pTSS region is calculated.

TPM-like calculation is performed to obtain a TPM-like normalized pTSS coverage (NPC-TPM) of each gene, to reduce an impact of a sequencing depth on data extraction and analysis.

$$NPC - {TPM}_{i} = \frac{{q}_{i} / {l}_{i}}{{\sum }_{j} ( {q}_{j} / {l}_{j} )} * 1 {0}^{6} = \frac{{q}_{i}}{{\sum }_{j} {q}_{j}} * 1 {0}^{6}$$

where NPC-TPMᵢ represents a TPM-like normalized pTSS coverage of a gene i, qᵢ represents an original read coverage at the pTSS region, lᵢ represents a transcript length (all being 2000), and Σⱼ(qⱼ/lⱼ) represents a sum of pTSS read coverages of all genes normalized based on the transcript length in one sample.

NPC-TPM values corresponding to all genes are input to the system as impact factors.

A feature selection module is configured to match high-risk pregnant women in a severe PAS group and high-risk pregnant women without occurrence of PAS at 1:1 in the training dataset by propensity scoring, then perform differential analysis on the impact factors by DESeq2, limma-voom, and a rank-sum test, and select an impact factor with a p value <0.05 obtained by using the three differential analysis methods or an impact factor with a p value <0.05 obtained by using at least two methods as a feature factor.

A feature factor discretization module is configured to implement a discretization strategy to enhance universality and clinical utility of the classifier for different sequencing platforms:

The optimal cutoff value of each feature factor is set to a NPC-TPM value with a maximum sum of sensitivity and specificity in the training dataset. A case in which a feature factor NPC-TPM is greater than a corresponding optimal cutoff value is set to 1; and a case in which a feature factor NPC-TPM is not greater than a corresponding optimal cutoff value is set to 0.

A model acquisition module is configured to input the selected feature factors to a machine learning feature selection process, for example, recursive feature elimination (RFE), and gradually construct a PAS prediction classifier by using a plurality of machine learning algorithms such as support vector machine (SVM)-linear kernel and SVM-gaussian kernel function (radial basis function, RBF).

PAS pregnant women set to 1 and non-PAS high-risk pregnant women set to 0 are input to the system for classifier training. To increase sensitivity of the classifier, the class_weight parameter is set to {0:0.1, 1:0.3}.

A disease risk assessment result of a to-be-predicted target is acquired, and k-fold cross-validation (k=10) is applied to enhance assessment robustness.

An optimal feature factor combination is extracted, and an optimal classifier and an assessment result thereof are output.

### BENEFICIAL EFFECTS OF THE PRESENT INVENTION

PAS is a collective term for a group of diseases characterized by abnormal placental adhesion or invasion into myometrium. The placenta of a fetus delivered from a PAS patient fails to detach normally, causing massive blood loss on a placental separation surface. This is a leading cause of critical and life-threatening obstetric conditions such as emergency hysterectomy, multiple organ failure, disseminated intravascular coagulation, and shock, and even perinatal mortality. In recent years, the incidence of PAS has risen annually due to increased cesarean sections, and development in intrauterine procedures and assisted reproductive technologies. Statistics indicate that PAS occurs in one of every 300 to 400 cases of pregnancy. Although PAS cases diagnosed prenatally using an existing clinical method usually involve severe invasion into the myometrium, they exhibit lower rates of emergency cesarean section, blood loss, and blood transfusions than PAS cases undiagnosed prenatally. Therefore, prenatal identification and perinatal management of PAS are critically important.

This study develops a classifier based on NPC of 23 genes, which can effectively predict a PAS risk in high-risk pregnant women based on non-invasive blood testing during early-to-mid pregnancy, demonstrating significant clinical application value. This innovative method holds promise for direct clinical practice, and provides scientific basis and practical guidance for early PAS diagnosis and treatment, demonstrating great clinical significance for early prediction of clinical outcomes of PAS pregnant women, referrals of high-risk pregnant women, multidisciplinary consultations, and reduction of risks in pregnant or lying-in women and perinatal infants.

### BRIEF DESCRIPTION OF THE DRAWINGS

FIG. 1 is a Venn diagram of differential gene screening in Example 1;

FIG. 2 is a Venn diagram of differential gene screening in Example 2;

FIG. 3 is a schematic diagram of performance of a 25-gene LR classifier obtained by inputting NPC-TPM values of 702 genes in Example 2; and

FIG. 4 is a schematic diagram of performance of a 23-gene SVM-RBF kernel classifier obtained by inputting NPC-TPM values of 702 genes in Example 2.

### DESCRIPTION OF THE EMBODIMENTS

The following further describes the present invention with examples, but the protection scope of the present invention is not limited thereto.

A person skilled in the art should understand that the examples of the present invention may be provided as a system or a computer program product. Therefore, the present invention may use a form of a hardware-only example, a software-only example, or an example with a combination of software and hardware. In addition, the present invention may use a form of a computer program product that is implemented on one or more computer-usable storage media that include computer-usable program code. The storage medium may be implemented by any type of volatile or non-volatile storage device or a combination thereof, such as a static random access memory (SRAM), an electrically erasable programmable read-only memory (EEPROM), an erasable programmable read-only memory (EPROM), a programmable read-only memory (PROM), a read-only memory (ROM), a magnetic memory, a flash memory, a magnetic disk, or an optical disc. These computer program instructions may alternatively be stored in a computer-readable memory that can instruct a computer or another programmable data processing device to work in a specific manner, so that instructions stored in the computer-readable memory generate an artifact that includes an instruction apparatus. The instruction apparatus implements a function of early identification of a placenta accreta spectrum risk in high-risk pregnant women.

### Example 1

PAS high-risk cohorts were established in a maternal and child health care hospital in city A (BGI sequencing platform) and in a maternal and child health care hospital in city B (Illumina sequencing platform). NIPT data and clinical information were prospectively collected from pregnant women who underwent non-invasive DNA screening and possessed high-risk factors of PAS (at least one of history of uterine surgery such as cesarean section and myomectomy, history of hysteroscopic surgery or intrauterine procedures, and pregnancy achieved via assisted reproductive technology). Follow-up was conducted to 28 days postpartum.

Exclusion criteria include: (1) non-live birth outcomes such as stillbirth and neonatal death; (2) fetal chromosomal abnormalities or developmental malformations; and (3) multiple gestations. Pregnant women with occurrence of PAS and pregnant women without occurrence of PAS were matched based on maternal age, gestational age at NIPT, fetal sex, and distribution of high-risk factors.

In the high-risk pregnant women who underwent NIPT on the BGI sequencing platform, 54 cases of PAS pregnant women and 157 cases of non-PAS high-risk pregnant women were included in a training dataset, 26 cases of PAS pregnant women and 71 cases of non-PAS high-risk pregnant women were included in an internal validation dataset, and 25 cases of PAS pregnant women and 77 cases of non-PAS high-risk pregnant women were included in a time validation dataset, for differential analysis and model training. In addition, 54 cases of PAS pregnant women and 162 cases of non-PAS high-risk pregnant women who underwent NIPT on the Illumina sequencing platform were included in an independent external validation dataset.

NIPT data from the enrolled pregnant women was annotated with cfDNA promoter nucleosome coverage profiles and aligned to human reference genome hg19. PCR duplicates were removed. Original read coverages at pTSS regions were calculated. The original read coverages were normalized via two methods to respectively obtain an NPC-RPKM value and an NPC-TPM value of each gene.

$$NPC - {RPKM}_{i} = \frac{{q}_{i}}{{l}_{i} * tmr} * {10}^{9}$$

$$NPC - {TPM}_{i} = \frac{{q}_{i} / {l}_{i}}{{\sum }_{j} ( {q}_{j} / {l}_{j} )} * 1 {0}^{6} = \frac{{q}_{i}}{{\sum }_{j} {q}_{j}} * 1 {0}^{6}$$

where NPC-RPKMᵢ represents an RPKM-like normalized pTSS coverage of a gene i, NPC-TPMᵢ represents a TPM-like normalized pTSS coverage of the gene i, qᵢ represents an original read coverage at the pTSS region, lᵢ represents a transcript length (all being 2000), tmr represents a total length of all genes, and Σⱼ(qⱼ/lⱼ) represents a sum of pTSS read coverages of all genes normalized based on the transcript length in one sample.

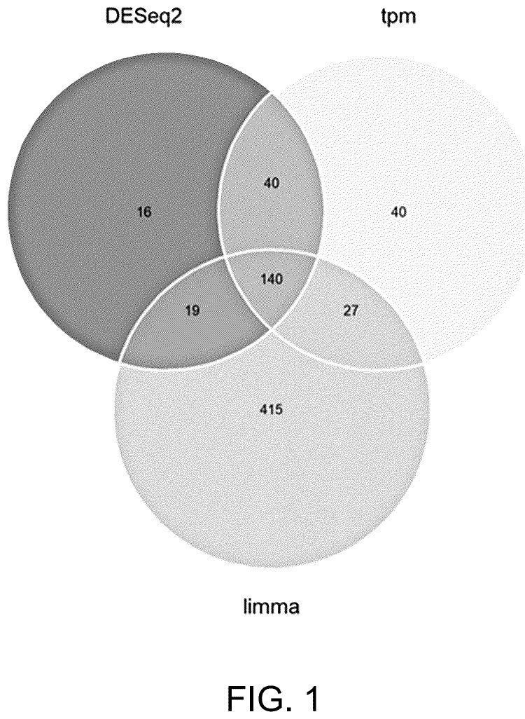

The PAS pregnant women and the non-PAS high-risk pregnant women in the training dataset were matched at 1:1 to obtain 54 cases of samples in each group. Genes with a p value <0.05 and log 2FC>0.5 that were obtained by using at least two of the three differential analysis methods: DESeq2, limma-voom, and a rank-sum test were selected, to obtain 226 differential pTSS coverage genes. FIG. 1 is a Venn diagram of differential gene screening.

To validate effects of models normalized via different methods, in this example, optimal cutoff values of the 226 feature factors were set to an NPC-RPKM value and an NPC-TPM value with a maximum sum of sensitivity and specificity in the training dataset. A case in which a feature factor NPC (NPC-RPKM or NPC-TPM) was greater than a corresponding optimal cutoff value was set to 1. A case in which a feature factor NPC (NPC-RPKM or NPC-TPM) was not greater than a corresponding optimal cutoff value was set to 0.

The feature factors obtained through screening were input to an RFE process, and a PAS prediction classifier was gradually constructed by using a plurality of machine learning algorithms. SVM-Linear kernel, SVM-RBF kernel, and LR were selected separately for classifier training. PAS pregnant women set to 1 and non-PAS high-risk pregnant women set to 0 were input to the system for classifier training. To increase sensitivity of the classifier, the class_weight parameter was set to {0:0.1, 1:0.3}. A disease risk assessment result of a to-be-predicted target was acquired, and k-fold cross-validation (k=10) was applied to enhance assessment robustness. Optimal feature factor combinations across different modeling methods were extracted, and an optimal classifier and an assessment result thereof were output.

For the NPC-RPKM value, an output result is a 23-gene SVM-Linear classifier. This classifier is poor in validation performance (AUC<0.5) in external validation sets on different platforms, as shown in Table 1.

TABLE 1

| | 23-gene SVM-Linear classifier obtained by | | | |
|---|---|---|---|---|
| | inputting NPC-RPKM values of 226 genes | | | |
| Dataset | AUC | Accuracy | Sensitivity | Specificity |
| Training dataset | 0.962 | 0.910 | 0.926 | 0.904 |
| Internal validation set | 0.910 | 0.907 | 0.808 | 0.944 |
| Time validation set | 0.787 | 0.754 | 0.831 | 0.792 |
| External validation set | 0.447 | 0.565 | 0.296 | 0.377 |

For the NPC-TPM value, an output result is a 31-gene SVM-Linear kernel classifier. This classifier is significantly improved in validation performance in the time validation set and the external validation set, indicating that the NPC-TPM value is more suitable for data from different platforms, as shown in Table 2.

TABLE 2

| | 31-gene SVM-Linear kernel classifier obtained | | | |
|---|---|---|---|---|
| | by inputting NPC-TPM values of 226 genes | | | |
| Dataset | AUC | Accuracy | Sensitivity | Specificity |
| Training dataset | 0.962 | 0.867 | 0.963 | 0.834 |
| Internal validation set | 0.819 | 0.763 | 0.846 | 0.732 |
| Time validation set | 0.899 | 0.794 | 0.880 | 0.766 |
| External validation set | 0.592 | 0.846 | 0.315 | 0.759 |

For the NPC-TPM value, output results are a 38-gene LR classifier and a 38-gene SVM-RBF kernel classifier, as shown in Table 3 and Table 4. These two classifiers are both improved (AUC>0.7) in validation performance in the external validation set, and the SVM-RBF kernel classifier has optimal performance. This indicates that the NPC-TPM discrete data suitable for different platforms is more consistent with the nonlinear SVM-RBF kernel model, and the SVM-RBF kernel is more suitable for constructing classifiers across different platforms.

TABLE 3

| | 38-gene LR classifier obtained by | | | |
|---|---|---|---|---|
| | inputting NPC-TPM values of 226 genes | | | |
| Dataset | AUC | Accuracy | Sensitivity | Specificity |
| Training dataset | 0.946 | 0.830 | 0.897 | 0.808 |
| Internal validation set | 0.797 | 0.714 | 0.771 | 0.695 |
| Time validation set | 0.732 | 0.628 | 0.744 | 0.589 |
| External validation set | 0.749 | 0.671 | 0.722 | 0.654 |

TABLE 4

| | 38-gene SVM-RBF kernel classifier obtained | | | |
|---|---|---|---|---|
| | by inputting NPC-TPM values of 226 genes | | | |
| Dataset | AUC | Accuracy | Sensitivity | Specificity |
| Training dataset | 0.967 | 0.872 | 0.962 | 0.842 |
| Internal validation set | 0.856 | 0.786 | 0.800 | 0.781 |
| Time validation set | 0.796 | 0.715 | 0.721 | 0.713 |
| External validation set | 0.856 | 0.773 | 0.870 | 0.741 |

### Example 2

Based on Example 1, a PAS high-risk cohort in a maternal and child health care hospital in city C (Life sequencing platform) was added. Inclusion and exclusion criteria, and a method for prospectively collecting sample data are the same as those in Example 1.

In the high-risk pregnant women who underwent NIPT on the BGI sequencing platform, 70 cases of PAS pregnant women and 210 cases of non-PAS high-risk pregnant women were included in a training dataset, and 35 cases of PAS pregnant women and 95 cases of non-PAS high-risk pregnant women were included in an internal validation dataset, for differential analysis and model training. In addition, 51 cases of PAS pregnant women and 163 cases of non-PAS high-risk pregnant women who underwent NIPT PLUS detection (with a higher sequencing depth) on the BGI sequencing platform in the maternal and child health care hospital in city A, 54 cases of PAS pregnant women and 162 cases of non-PAS high-risk pregnant women enrolled in the maternal and child health care hospital in city B, and 55 cases of PAS pregnant women and 165 cases of non-PAS high-risk pregnant women enrolled in the maternal and child health care hospital in city C were included respectively in three independent external validation datasets (an NIPT PLUS dataset, an Illumina validation set, and a Life validation set).

NIPT data from the enrolled pregnant women was annotated with cfDNA promoter nucleosome coverage profiles by using the method as described in Example 1. NPC-TPM values were input to a classifier training system.

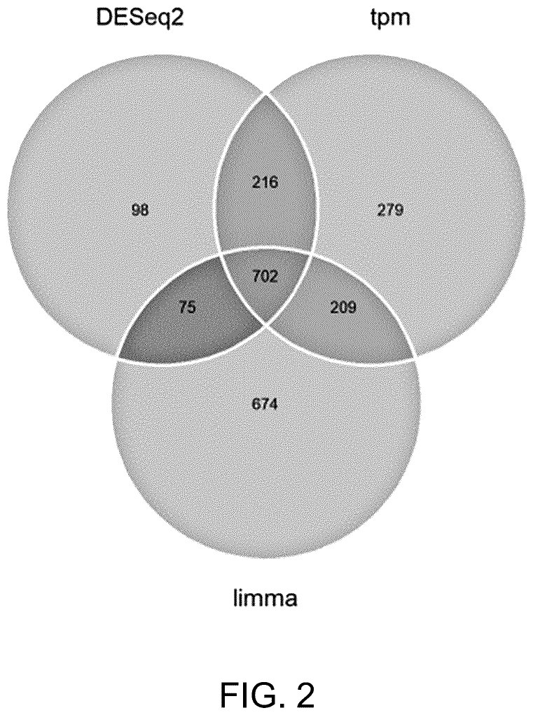

The pregnant women in a severe PAS group and the non-PAS high-risk pregnant women in the training dataset were matched at 1:1 to obtain 35 cases of samples in each group. Genes with a p value <0.05 that were obtained by using three differential analysis methods: DESeq2, limma-voom, and a rank-sum test were selected, to obtain 702 differential pTSS coverage genes. FIG. 2 is a Venn diagram of differential gene screening.

Optimal cutoff values of the 702 feature factors were set to an NPC-TPM value with a maximum sum of sensitivity and specificity in the training dataset. A case in which a feature factor NPC (NPC-TPM) was greater than a corresponding optimal cutoff value was set to 1. A case in which a feature factor NPC (NPC-TPM) was not greater than a corresponding optimal cutoff value was set to 0.

The feature factors obtained through screening were input to an RFE process, and a PAS prediction classifier was gradually constructed by using a plurality of machine learning algorithms. SVM-RBF kernel and LR were selected separately for classifier training.

PAS pregnant women set to 1 and non-PAS high-risk pregnant women set to 0 were input to the system for classifier training. To increase sensitivity of the classifier, the class_weight parameter was set to {0:0.1, 1:0.3}.

A disease risk assessment result of a to-be-predicted target was acquired, and k-fold cross-validation (k=10) was applied to enhance assessment robustness.

Optimal feature factor combinations across different modeling methods were extracted, and an optimal classifier and an assessment result thereof were output.

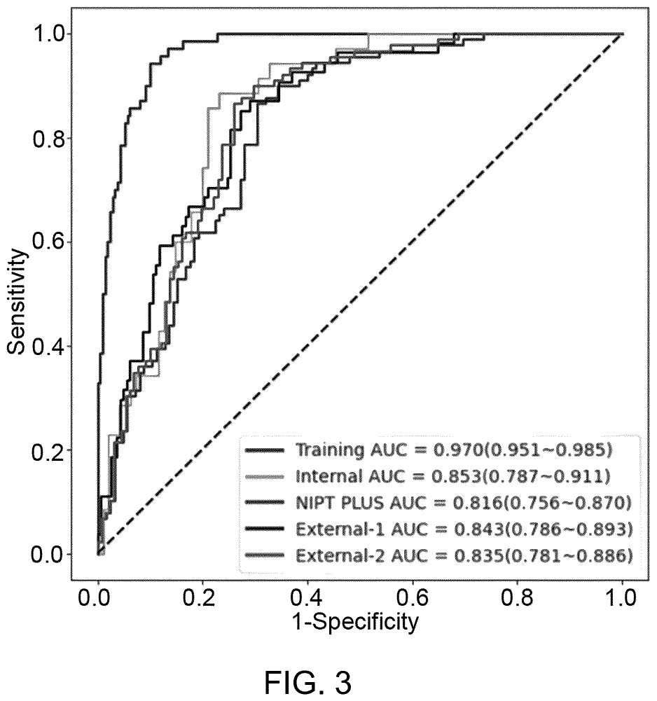

By using the LR training method, an output result is a 25-gene LR classifier. The AUC, accuracy, sensitivity, and specificity of this classifier were calculated in the training dataset, the internal validation dataset, and the external validation dataset, as shown in Table 5 and FIG. 3.

TABLE 5

| | 25-gene LR classifier obtained by | | | |
|---|---|---|---|---|
| | inputting NPC-TPM values of 702 genes | | | |
| Dataset | AUC | Accuracy | Sensitivity | Specificity |
| Training dataset | 0.970 | 0.871 | 0.971 | 0.838 |
| | (0.951-0.985) | | | |
| Internal validation | 0.853 | 0.792 | 0.800 | 0.789 |
| set | (0.787-0.911) | | | |
| PLUS validation set | 0.816 | 0.752 | 0.831 | 0.696 |
| | (0.756-0.870) | | | |
| External validation | 0.843 | 0.759 | 0.852 | 0.728 |
| set 1 | (0.786-0.893) | | | |
| External validation | 0.835 | 0.777 | 0.831 | 0.740 |
| set 2 | (0.781-0.886) | | | |

By using the SVM-RBF kernel method, an output result is a 23-gene SVM-RBF kernel classifier. Discretization thresholds for each gene are shown in Table 6.

TABLE 6

| | Average | Average | | |
|---|---|---|---|---|
| | NPC value | NPC value | | |
| | in the | in the | Discretized | Optimal |
| | PAS | control | single-gene | cutoff |
| Gene | group | group | AUC value | value |
| TMEM147.AS1 | 43.19 | 32.95 | 0.612 | 45.46 |
| MIR184 | 44.73 | 37.85 | 0.598 | 46.77 |
| NSD2 | 11.57 | 16.41 | 0.597 | 15.68 |
| KRT5 | 41.72 | 51.76 | 0.590 | 47.10 |
| SLC16A12.AS1 | 36.08 | 44.76 | 0.586 | 43.83 |
| LINC00964 | 38.03 | 46.51 | 0.575 | 41.89 |
| NGDN | 22.79 | 28.37 | 0.574 | 26.80 |
| LYZL2 | 39.77 | 27.84 | 0.566 | 36.72 |
| ALG1L2 | 36.91 | 45.08 | 0.566 | 37.76 |
| PACRG.AS3 | 39.00 | 31.25 | 0.564 | 43.68 |
| KDSR | 45.66 | 38.77 | 0.563 | 32.27 |
| TADA3 | 39.23 | 32.99 | 0.562 | 38.59 |
| ABHD1 | 34.89 | 28.45 | 0.557 | 26.55 |
| FAM157C | 38.38 | 30.42 | 0.544 | 23.90 |
| MYT1L | 39.40 | 47.20 | 0.543 | 29.24 |
| LOC107987394 | 51.25 | 43.29 | 0.541 | 37.49 |
| LOC105371998 | 33.62 | 40.42 | 0.539 | 30.00 |
| LANCL2 | 30.05 | 24.23 | 0.532 | 30.12 |
| LINC00390 | 51.16 | 42.21 | 0.531 | 45.28 |
| LOC644090 | 45.52 | 38.62 | 0.523 | 47.23 |
| MIR4802 | 37.53 | 44.81 | 0.500 | 40.41 |
| EYS | 34.99 | 42.56 | 0.496 | 36.81 |
| SAP30L.AS1 | 33.09 | 40.24 | 0.479 | 34.30 |

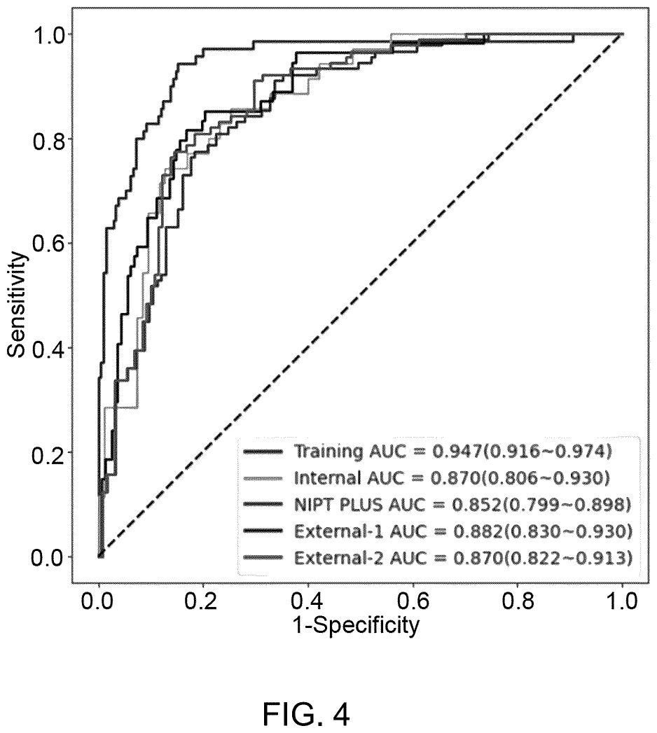

The AUC, accuracy, sensitivity, and specificity of this classifier were calculated in the training dataset, the internal validation dataset, and the external validation dataset, as shown in Table 7 and FIG. 4. It can be learned that the SVM classifier has a small quantity of genes and is better than the LR classifier in prediction performance. 35 cases of pregnant women in a severe PAS group and 35 cases of non-PAS high-risk pregnant women were selected and matched at 1:1. Then, a 702-feature factor set was obtained through screening, which is better than a 226-feature factor set obtained through screening from 54 cases of PAS pregnant women and 54 cases of non-PAS high-risk pregnant women. This result indicates that accurate early prediction can be implemented on PAS by integrating expression levels of the 23 genes: ABHD1, ALG1L2, EYS, FAM157C, KDSR, KRT5, LANCL2, LINC00390, LINC00964, LOC105371998, LOC107987394, LOC644090, LYZL2, MIR184, MIR4802, MYT1L, NGDN, NSD2, PACRG.AS3, SAP30L.AS1, SLC16A12.AS1, TADA3, and TMEM147.AS1, and a detection method can be designed with high sensitivity and high specificity, holding promise for preparation of a diagnostic kit widely used in clinical diagnosis, advancing personalized medicine and precision treatment.

TABLE 7

| | 23-gene SVM-RBF kernel classifier obtained | | | |
|---|---|---|---|---|
| | by inputting NPC-TPM values of 702 genes | | | |
| Dataset | AUC | Accuracy | Sensitivity | Specificity |
| Training dataset | 0.947 | 0.868 | 0.914 | 0.852 |
| | (0.912-0.974) | | | |
| Internal validation | 0.870 | 0.792 | 0.800 | 0.789 |
| set | (0.801-0.927) | | | |
| PLUS validation set | 0.852 | 0.776 | 0.831 | 0.736 |
| | (0.800-0.898) | | | |
| External validation | 0.882 | 0.759 | 0.852 | 0.728 |
| set 1 | (0.824-0.928) | | | |
| External validation | 0.870 | 0.795 | 0.831 | 0.771 |
| set 2 | (0.822-0.913) | | | |

It is noted that the dataset division module, the impact factor extraction module, the feature selection module, the feature factor discretization module, and the model acquisition module may be implemented with the help of software and a necessary general-purpose hardware platform. For example, in an exemplary embodiment of the present invention, the classifier training system may also include an output apparatus, an input apparatus, a storage apparatus, and a control circuit. Instructions and functions executed by the dataset division module, the impact factor extraction module, the feature selection module, the feature factor discretization module, and the model acquisition module may run in the control circuit. For example, the control circuit is a central processing unit (CPU), another programmable general-purpose or special-purpose microprocessor, a digital signal processor (DSP), a programmable controller, an application-specific integrated circuit (ASIC), another similar element, or a combination thereof, and can run the dataset division module, the impact factor extraction module, the feature selection module, the feature factor discretization module, and the model acquisition module, to implement the function of constructing the classifier for early identification of a placenta accreta spectrum risk in high-risk pregnant women. The classifier for early identification of a placenta accreta spectrum risk in high-risk pregnant women provided in this application may be implemented with the help of software and a necessary general-purpose hardware platform. A computer software product is stored in a storage medium (such as ROM/RAM, a magnetic disk, or an optical disc), and includes several instructions for a terminal device (which may be a mobile phone, a computer, a server, a controlled terminal, or a network device) to perform the method in each example of this application.

The specific examples described herein are merely illustrative examples of the spirit of the present invention. A person skilled in the art to which the present invention pertains may make various modifications or additions or similar replacements to the specific examples, without departing from the spirit of the present invention or exceeding the scope defined by the appended claims.

1. A classifier training system for early identification of a placenta accreta spectrum risk in high-risk pregnant women, comprising:
a dataset division module, configured to extract pre-collected medical data of placenta accreta spectrum (PAS) high-risk pregnant women, and divide the medical data into a discovery dataset, a training dataset, an internal validation dataset, and an external validation dataset;
an impact factor extraction module, configured to perform annotation on pre-collected non-invasive prenatal testing (NIPT) data of the PAS high-risk pregnant women with genome-wide cell-free DNA promoter nucleosome coverage profiles and perform feature extraction, calculate original read coverages at pTSS regions, and perform TPM-like normalization on the original read coverages at the pTSS regions to obtain TPM-like normalized pTSS coverages NPC-TPM, wherein NPC-TPM corresponding to each gene is used as an impact factor;
a feature selection module, configured to screen impact factors of high-risk pregnant women with occurrence of PAS and high-risk pregnant women without occurrence of PAS in the discovery dataset, to determine feature factors;
a feature factor discretization module, configured to determine an optimal cutoff value of each feature factor, and perform discretization on the feature factors based on the optimal cutoff value;
a model acquisition module, configured to input the determined feature factors to a recursive feature elimination (RFE) process, gradually construct a PAS prediction classifier by using a plurality of machine learning algorithms; acquire a disease risk assessment result of a to-be-predicted target, apply k-fold cross-validation to enhance assessment robustness; and extract an optimal feature factor combination, and output an optimal classifier and an assessment result thereof, wherein in the model acquisition module, the optimal feature factor combination comprises the following target genes: ABHD1, ALG1L2, EYS, FAM157C, KDSR, KRT5, LANCL2, LINC00390, LINC00964, LOC105371998, LOC107987394, LOC644090, LYZL2, MIR184, MIR4802, MYT1L, NGDN, NSD2, PACRG.AS3, SAP30L.AS1, SLC16A12.AS1, TADA3, and TMEM147.AS1.

2. The system according to claim 1, wherein in the dataset division module, pregnant women with occurrence of PAS and pregnant women without occurrence of PAS are matched based on maternal age, gestational age at NIPT, fetal sex, and distribution of high-risk factors, samples from a primary center are randomly divided into the training dataset and the internal validation dataset, samples from each sub-center are used as the external validation dataset, and matching is performed on the training dataset at 1:1 based on the gestational age at NIPT and the fetal sex to obtain the discovery dataset.

3. The system according to claim 1, wherein in the impact factor extraction module, NIPT data is aligned to human reference genome hg19 by using a sequence alignment algorithm, PCR duplicates are removed, a region from −1000 bp to +1000 bp around a transcription start site (TSS) is determined as a promoter region pTSS, and the original read coverage at the pTSS region is calculated.

4. The system according to claim 3, wherein in the impact factor extraction module, TPM-like normalization is performed on the original read coverage at the pTSS region, wherein NPC-TPM is obtained through the following formula:
$$NPC - {TPM}_{i} = \frac{{q}_{i} / {l}_{i}}{{\sum }_{j} ( {q}_{j} / {l}_{j} )} * 1 {0}^{6} = \frac{{q}_{i}}{{\sum }_{j} {q}_{j}} * 1 {0}^{6}$$
wherein NPC-TPMᵢ represents a TPM-like normalized pTSS coverage of a gene i, qᵢ represents an original read coverage at the pTSS region, lᵢ represents a transcript length, and Σⱼ(qⱼ/lⱼ) represents a sum of pTSS read coverages of all genes normalized based on the transcript length in one sample.

5. The system according to claim 1, wherein in the feature selection module, the impact factors of the high-risk pregnant women with occurrence of PAS and the high-risk pregnant women without occurrence of PAS in the discovery dataset are subjected to three differential analysis methods: DESeq2, limma-voom, and a rank-sum test, to obtain impact factors with a p value of less than 0.05 as the feature factors.

6. The system according to claim 1, wherein in the feature factor discretization module, to enhance universality and clinical utility of the classifier for different sequencing platforms, the optimal cutoff value of each feature factor is set to a NPC-TPM value with a maximum sum of sensitivity and specificity in the training dataset; a case in which a feature factor NPC-TPM is greater than a corresponding optimal cutoff value is set to 1; and a case in which a feature factor NPC-TPM is not greater than a corresponding optimal cutoff value is set to 0.

7. The system according to claim 1, wherein in the model acquisition module, the PAS prediction classifier is gradually constructed by using the plurality of machine learning algorithms, and the classifier is trained separately by logistic regression (LR), and support vector machines (SVM) with linear and RBF kernels.

8. The system according to claim 1, wherein in the model acquisition module:
support vector machine (SVM)-RBF kernel is selected for the optimal classifier.

9. A classifier for early identification of a placenta accreta spectrum risk in high-risk pregnant women, wherein obtained through the training system according to claim 1.

10. Use of a reagent for detecting expression of genes ABHD1, ALG1L2, EYS, FAM157C, KDSR, KRT5, LANCL2, LINC00390, LINC00964, LOC105371998, LOC107987394, LOC644090, LYZL2, MIR184, MIR4802, MYT1L, NGDN, NSD2, PACRG.AS3, SAP30L.AS1, SLC16A12.AS1, TADA3, and TMEM147.AS1 in preparation of a diagnostic kit for early identification of a placenta accreta spectrum risk in high-risk pregnant women.

11. A classifier for early identification of a placenta accreta spectrum risk in high-risk pregnant women, wherein obtained through the training system according to claim 2.

12. A classifier for early identification of a placenta accreta spectrum risk in high-risk pregnant women, wherein obtained through the training system according to claim 3.

13. A classifier for early identification of a placenta accreta spectrum risk in high-risk pregnant women, wherein obtained through the training system according to claim 4.

14. A classifier for early identification of a placenta accreta spectrum risk in high-risk pregnant women, wherein obtained through the training system according to claim 5.

15. A classifier for early identification of a placenta accreta spectrum risk in high-risk pregnant women, wherein obtained through the training system according to claim 6.

16. A classifier for early identification of a placenta accreta spectrum risk in high-risk pregnant women, wherein obtained through the training system according to claim 7.

17. A classifier for early identification of a placenta accreta spectrum risk in high-risk pregnant women, wherein obtained through the training system according to claim 8.
